(19) United States
(12) Patent Application Publication
(10) Pub. No.: US 2014/0106000 A1
(43) Pub. Date: Apr. 17, 2014

(54) METHOD FOR FILLING BONE CAVITY FORMATIONS WITH CALCIUM

(71) Applicants: Villiori Ivanovich Strukov, Penza (RU); Vyacheslav Nikolaevich Trifonov, Zarechnii (RU); Yuliya Anatolevna Elistratova, Moscow (RU); Konstatin Gennadevich Elistratov, Penza (RU); Natalya Vyacheslavovna Kurus, Penza (RU)

(72) Inventors: Villiori Ivanovich Strukov, Penza (RU); Vyacheslav Nikolaevich Trifonov, Zarechnii (RU); Yuliya Anatolevna Elistratova, Moscow (RU); Konstatin Gennadevich Elistratov, Penza (RU); Natalya Vyacheslavovna Kurus, Penza (RU)

(21) Appl. No.: 14/123,136

(22) PCT Filed: Jan. 31, 2012
(86) PCT No.: PCT/RU2012/000049

§ 371 Date: Nov. 29, 2013

(30) Foreign Application Priority Data
May 31, 2011 (RU) 2011121932

(51) Int. Cl. A61K 35/64; A61K 33/06

(52) U.S. Cl. CPC A61K 35/642; A61K 33/06
USPC 424/539

(57) ABSTRACT

A method is proposed for filling cavity formations in metaphyseal (trabecular) bone sections with calcium and for preventing secretions of calcium therefrom. The proposed method consists in supplying drone brood to an organism in combination with supplying calcium to the organism. Existing methods for saturating an organism with calcium do not achieve the desired result.

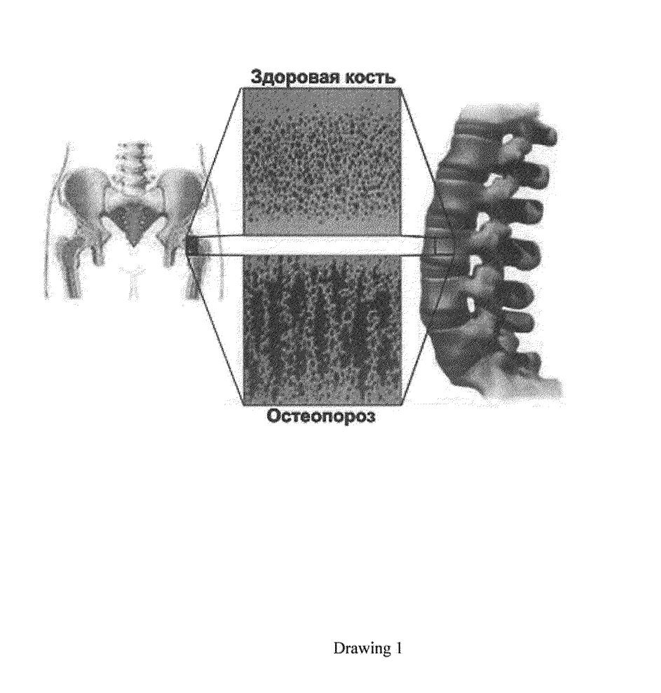

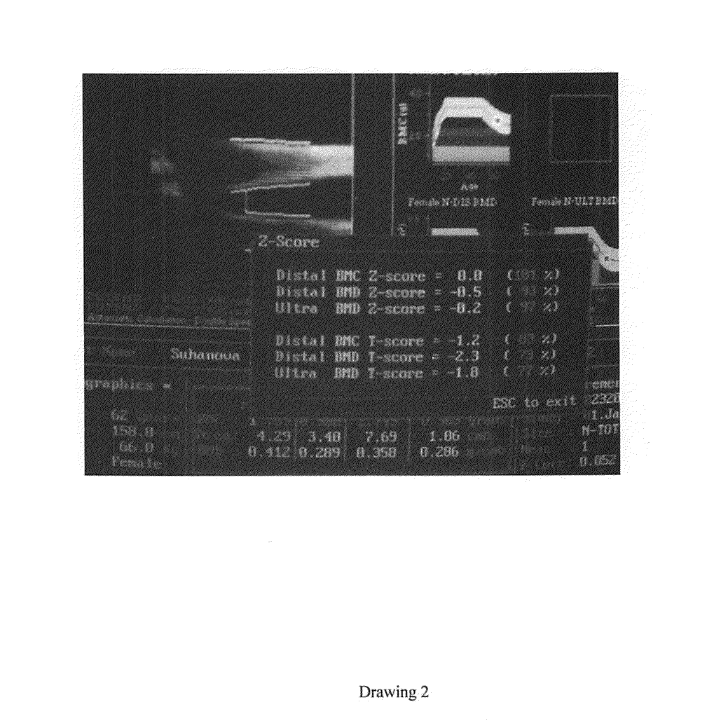

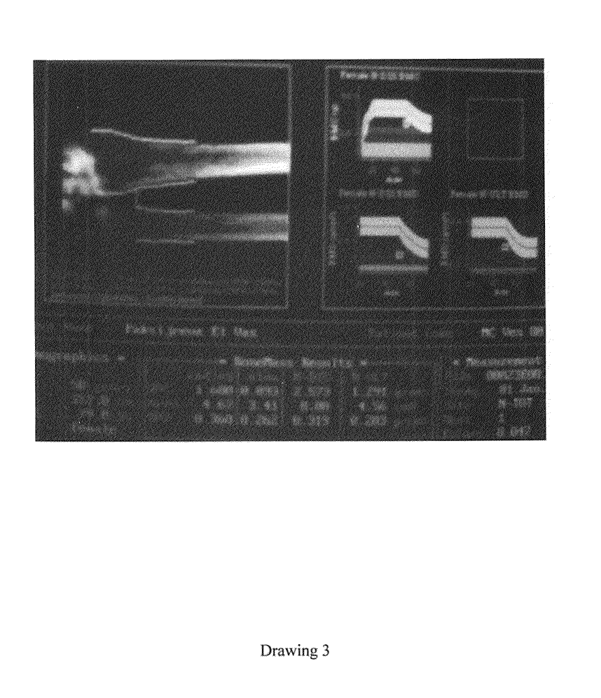

The invention relates to medicine, in particular to methods for combating the effects of calcium deficiency in the skeletal system of the body, which manifests in the form of an increase in the quantity and size of cavities in metaphyseal (trabecular) bone sections and for preventing such manifestations of calcium deficiency.

The existing methods for preventing and treating calcium deficiency in the body aim to saturate bone tissue with calcium. To that end, various calcium-containing preparations as well as vitamin D and hormonal preparations (such as testosterone) are used.

These methods have a number of significant disadvantages. Although calcium is not a toxic substance and no maximum allowable doses for administration have been established for it, prolonged use of significant quantities of calcium may increase the risk of stroke. When calcium preparations are being used, calcium can be supplied to other organs and systems as well as to bones, and in particular, can calcify calculi in the kidneys, which in turn leads to kidney function issues.

Similarly, there is a risk of exceeding the safe quantity of vitamin D in the body. In several cases the use of hormonal preparations is undesirable or completely excluded for medical reasons. Artificially increasing testosterone in females (even for apparently healthy females) is generally undesirable. It is also important to consider the fact that using calcium-containing preparations either separately or in combination with vitamins and/or hormones, in addition to all of the above-mentioned side effects and contra-indications, only makes it possible to increase mineralisation of bone tissue. Existent and newly-formed cavities in metaphyseal (trabecular) bone sections remain unchanged or the quantity and sizes thereof may continue to increase. These cavities reduce bone strength and increase the risk of injury at various loads.

While the applicant regularly takes part in research in this field, they are unaware of any methods that would provide for the desired result.

The current methods for increasing the calcium content in bone tissue in the body makes it possible to increase bone mineralisation but not to fill cavities therein.

The proposed solution aims to reduce and eliminate, where possible, existing cavities and to keep new cavities from forming. The desired result is achieved by providing for the supply of drone brood in combination with calcium to a body.

As experiments have shown, regular drone brood treatment in combination with supplying calcium to a body provides for the desired results.

So as to have a better visual understanding of the area for using this solution, an illustration is provided in drawing 1, which shows the location of cavities in the bone; showing both healthy bone and bone in a body that has been diagnosed with osteoporosis.

Drawing 2 shows the result of an X-ray examination prior to the commencement of treatment.

Two black cavities can be seen at the top and at the bottom of the bone.

Drawing 3 shows the results of an X-ray examination after a course of treatment. The cavities have been filled and have become blue.

The research was carried out using an X-ray absorption method on NORLAND, OSTEOMETR DTX-100 and 200 apparatuses on a group of 10,000 patients.

The calcium in the cavities is predominantly in the form of calcium hydroxyapatite.

In addition to providing for the supply of calcium to the body in common food products, various calcium-containing preparations can be used including calcium carbonate, calcium citrate, calcium gluconate, calcium aspartate, calcium ascorbate, calcium amino chelate, calcium fumarate, calcium succinate, calcium phosphate, and calcium citrate, any one of these or any combination thereof.

During the experimental confirmation of the results, drone brood was given to patients orally in powder form, tablet form or capsule form either as the individual active agent or in combination with calcium-containing compounds.

The desired result can be achieved more quickly or more slowly depending on the individual properties of a particular body, the amount of calcium required in food and the quantity and size of the cavities, but the result is achieved in any case. It has been seen that 100% of patients achieved practically significant results over an extended course of treatment (nine months).

1. The method for filling cavities in metaphyseal (trabecular) bone sections with calcium and for preventing the secretion of calcium therefrom consists in providing for the supply of drone brood in combination with calcium to an organism.

2. The method according to claim 1, in which drone brood is orally supplied to a body in powder form, tablet form or capsule form.

3. The method according to claim 1, in which drone brood is supplied to a body in combination with calcium-containing preparations.

4. The method according to claim 3, in which calcium-containing preparations are one of the following groups of preparations or any combination thereof: calcium carbonate, calcium citrate, calcium gluconate, calcium aspartate, calcium ascorbate, calcium amino chelate, calcium fumarate, calcium succinate, calcium phosphate, and calcium citrate.
